(19) United States
(12) Patent Application Publication
(10) Pub. No.: US 2026/0183526 A1
(43) Pub. Date: Jul. 2, 2026

(54) Recirculation Connector for Hemodialysis with Integrated Clamping Mechanism

(71) Applicant: Fresenius Medical Care Holdings, Inc., Waltham, MA (US)

(72) Inventors: Jose Omar Aguilar Flores, Mission, TX (US); Diego Suarez del Real Pena, Mission, TX (US); Sergio Rieboles Casados, Reynosa (MX); Victoria Kihabet Salinas Cornejo, Reynosa (MX)

(21) Appl. No.: 19/004,926

(22) Filed: Dec. 30, 2024

(51) Int. Cl. A61M 39/10; A61M 1/14; A61M 39/00; A61M 39/20

(52) U.S. Cl. CPC A61M 39/10; A61M 39/20; A61M 1/14; A61M 2039/0009

(57) ABSTRACT

In one aspect a medical fluid connector functions as both a recirculation connector to connect arterial and venous lines during priming of an extracorporeal blood circuit as well as a clamp to occlude a fluid tube. In one aspect, the connector includes a tubular body with luer connections and a clamping arm that extends longitudinally along a tubular body to apply clamping force.

### TECHNICAL FIELD

This disclosure relates to fluid connectors, and more particularly to medical fluid connectors such as used in connection with dialysis treatments.

### BACKGROUND

Medical devices, such as dialysis machines, are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate, or dialysis solution. The membranous lining of the patient's peritoneum acts as a natural semi-permeable membrane that allows diffusion and osmosis exchanges to take place between the solution and the blood stream. Automated peritoneal dialysis machines, also called PD cyclers, are designed to control the entire peritoneal dialysis process so that it can be performed at home, usually overnight, without clinical staff in attendance.

In some traditional hemodialysis treatments, separate recirculation connectors and hemostats are utilized, each serving distinct roles in the process. The recirculation connector is employed during the priming stage to facilitate the connection between venous and arterial lines, ensuring the removal of air and particulates from the system as priming fluid is pushed through both tubes sequentially. In some systems, the recirculation connector creates a fluid pathway between an end of the arterial line and a an end of a venous line to allow the priming fluid to flow therethrough. After priming, each line is clamped closed near the site of the recirculation connector, and the recirculation connector is then removed. After removal of the recirculation connector, the ends of the lines are connected to their respective positions (e.g., patient arterial and venous accesses, respectively) for the treatment. Hemostats, on the other hand, are used to occlude fluid-lines during treatment to prevent air introduction and blood leaks. However, the use of multiple components (i.e., recirculation connectors and hemostats) can lead to increased complexity and a higher risk of operational errors. This complexity underscores the need for a more integrated solution that can streamline the hemodialysis process.

### SUMMARY

In one aspect, a method includes fluidly connecting a first end of an arterial blood line to a first end of a venous blood line via a recirculation connector, where the arterial and venous blood lines are part of an extracorporeal blood circuit of a dialysis machine. The method further includes flowing a priming fluid through the extracorporeal blood circuit such that the priming fluid either (a) flows from the arterial line to the venous line via the recirculation connector or (b) flows from the venous line to the arterial line via the recirculation connector. After flowing the priming fluid through the extracorporeal blood circuit, the method includes removing the recirculation connector. The method then includes occluding a flow path in the extracorporeal blood circuit by using the recirculation connector to apply a clamping force to an outer surface of a portion of the arterial line or the venous line.

The recirculation connector may include a clamping arm movable between an open position and a closed position, and the occluding may include moving the clamping arm from the open position to the closed position.

The method may also include latching the clamping arm into the closed position while applying the clamping force.

The method may further include unlatching the clamping arm to release the clamping force, removing the recirculation connector from the portion of the arterial line or venous line, and using the recirculation connector to apply a clamping force to another portion of the venous line or the arterial line.

The recirculation connector may include a tubular body, and the clamping arm may extend parallel to a longitudinal axis of the tubular body.

In another aspect, a medical fluid connector may include a tubular body having a first end, a second end, and an interior flow path extending along a longitudinal axis of the tubular body between the first end and the second end. The medical fluid connector further includes a first luer connector at the first end and configured to connect to an arterial line of an extracorporeal blood circuit, and a second luer connector at the second end and configured to connect to a venous line of an extracorporeal blood circuit. The medical fluid connector also includes a clamping portion extending from an exterior surface of the tubular body and including a clamping rib, the clamping portion movable relative to the tubular body between an open position and a closed position. When the clamping portion is in the open position, the medical fluid connector is configured to receive a medical tube between the clamping rib and the exterior surface of the tubular body such that the medical tube is oriented transversely to the longitudinal axis of the tubular body. When the clamping portion is in the closed position, the clamping rib extends parallel to the longitudinal axis of the tubular body and the medical fluid connector is configured to occlude the medical tube by applying a clamping force to the medical tube between the clamping rib and the exterior surface of the tubular body.

The medical fluid connector may further include a latch configured to maintain the clamping portion in the closed position.

The latch may include a protrusion on the first arm configured to engage with a corresponding recess on the second arm.

The first and second luer connectors may be female luer connectors.

The tubular body, the first luer connector, the second luer connector, and the clamping portion may be integrally formed as a single monolithic molded component.

The clamping portion may be configured to be repeatedly opened and closed.

The medical fluid connector may further include an end cap configured to cover the first luer connector or the second luer connector, and a tether having a first end connected to the end cap and a second end connected to the tubular body.

In another aspect, an extracorporeal line set for dialysis includes a venous blood line, an arterial blood line, and a recirculation connector. The recirculation connector includes a tubular body having a first end, a second end, and an interior flow path extending along a longitudinal axis of the tubular body between the first end and the second end. The recirculation connector further includes a first luer connector at the first end and configured to connect to the arterial line, and a second luer connector at the second end and configured to connect to the venous line. The recirculation connector also includes a clamping portion extending from an exterior surface of the tubular body and including a clamping rib, where the clamping portion is movable relative to the tubular body between an open position and a closed position. When the clamping portion is in the open position, the medical fluid connector is configured to receive a portion of the venous line or a portion of the arterial line between the clamping rib and the exterior surface of the tubular body such that the portion of the venous line or the portion of the arterial line is oriented transversely to the longitudinal axis of the tubular body. When the clamping portion is in the closed position, the clamping rib extends parallel to the longitudinal axis of the tubular body and the medical fluid connector is configured to occlude the portion of the venous line or the portion of the arterial line by applying a clamping force to the portion of the venous line or the portion of the arterial line between the clamping rib and the exterior surface of the tubular body.

Other aspects, features, and advantages will be apparent from the description, the drawings, and the claims.

### DESCRIPTION OF DRAWINGS

FIG. 1 shows a schematic of a blood line tubing set.

FIG. 2 shows a side view of a recirculation connector.

FIG. 3 shows another side view of the recirculation connector of FIG. 2.

FIG. 4 shows the recirculation connector of FIG. 2 with a clamp in a closed position.

FIG. 5 shows the recirculation connector of FIG. 2 with the clamp in the closed position while occluding a blood line.

FIG. 6 shows the recirculation connector and blood line of FIG. 5 showing the blood line in cross-section.

FIG. 7 shows a cross-sectional view of the recirculation connector and blood line of FIG. 5 taken along section A-A.

Like reference symbols in the various drawings indicate like elements.

### DETAILED DESCRIPTION

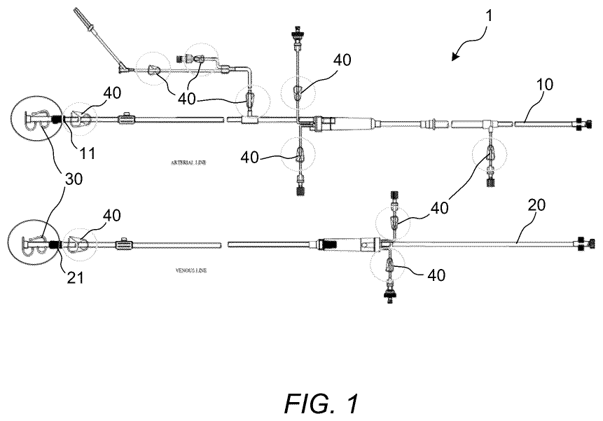

FIG. 1 shows a schematic representation of a blood line tubing set 1 utilized in hemodialysis treatment. As shown in the figure, the blood line tubing set 1 includes an arterial blood line 10 and a venous blood line 20. The arterial and venous blood lines 10 and 20 serve as conduits for blood flow during the hemodialysis process, facilitating the removal of waste products and excess fluids from the patient's bloodstream. As described herein, a recirculation connector 30 is depicted as facilitating the connection between the arterial and venous lines 10 and 20. This recirculation connector 30 is employed during the priming stage of hemodialysis, where it enables the circulation of saline solution through the system to remove air prior to the commencement of treatment.

The arterial blood line 10 is configured to convey blood from the patient to the dialysis apparatus, where impurities are filtered out. Conversely, the venous blood line 20 is designed to return the filtered blood back to the patient, completing the extracorporeal loop for hemodialysis treatment. The term “arterial blood line,” as used herein, generally refers to a conduit that transports blood from the patient to a medical device for processing, such as a dialysis machine. Examples of arterial blood lines may include flexible PVC tubing and silicone arterial lines. Similarly, the term “venous blood line” refers to a conduit responsible for returning processed blood to the patient. Examples of venous blood lines include venous return tubes and silicone venous lines.

The recirculation connector 30 plays a role during the priming stage of hemodialysis treatment by serving as a bridge that connects the arterial blood line 10 and the venous blood line 20. This connection is used for the recirculation of a priming fluid (e.g., saline solution), which is performed to purge air from the blood circuit, in this example including the arterial line 10, venous line 20 and a dialyzer, before the initiation of the hemodialysis procedure. The term “recirculation connector,” as used herein, generally refers to a component that facilitates the interconnection of blood lines to enable the fluid recirculation necessary for system preparation. During the priming process, the recirculation connector 30 ensures that the saline solution circulates between the arterial blood line 10 and the venous blood line 20, maintaining a closed system that prevents contamination and/or air entry and prepares the arterial blood line 10 and the venous blood line 20 for patient use.

Tubing clamps 40, as depicted in FIG. 1, are positioned along portions of the blood line tubing set 1 to facilitate control over fluid flow during hemodialysis treatment and priming. The function of each the tubing clamp 40 is to occlude a respective tube, thereby regulating the fluid dynamics within the hemodialysis system and preventing air introduction and potential leaks or other intended flow of blood or other fluid.

In some examples, after the priming fluid flows through the arterial blood line 10, recirculation connector 30, and venous blood line 20 to remove the air therein during a priming procedure, the tubing clamps 40 may be closed to occlude any flow of the priming fluid from the arterial blood line 10 through the arterial line patient connector 11 or from the venous line 20 through the venous patient connector 21. This prevents reintroduction of air into the arterial line 10 and venous line 20 before these lines are connected to the patient blood access (e.g., via an arteriovenous fistula or other patient blood access) via connectors 11 and 21. With the tubing clamps 40 remaining closed, the recirculation connector 30 is removed, and the respective patient connectors 11 and 21 are connected to the patient blood access. After successful connection, the tubing clamps 40 may be opened to allow blood to flow between the patient and the arterial and venous lines 10 and 20.

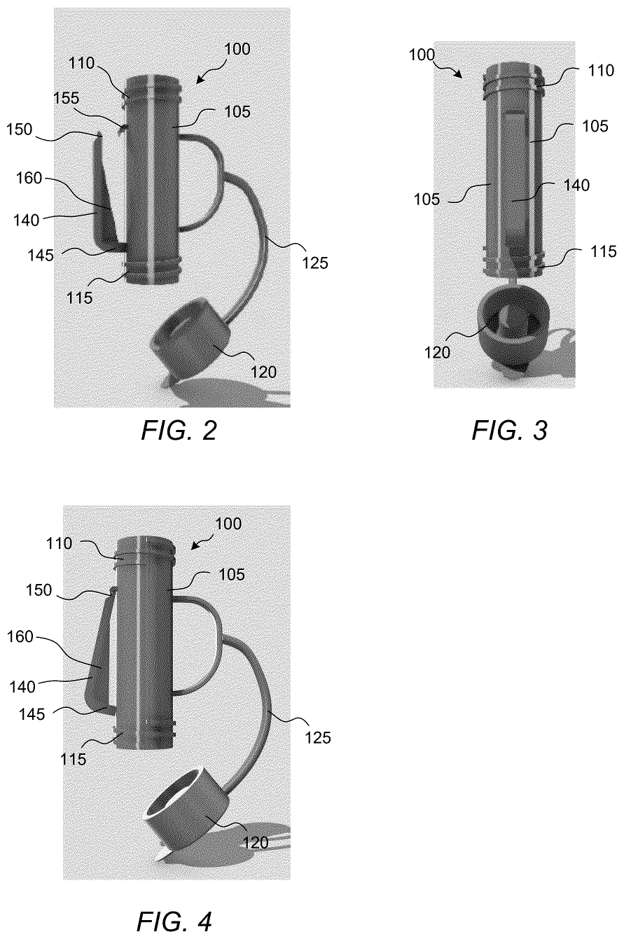

Referring to FIGS. 2 to 7, a recirculation connector 100 is provided for use in place of the recirculation connector 30 of FIG. 1. In contrast to the recirculation connector 30, which has no further use after priming and is therefore discarded, the recirculation connector 100 provides additional continued functionality by integrating recirculation and occlusion capabilities within a single device.

As shown in FIG. 2, the tubular main body 105 serves as the structural base of the recirculation connector 100, facilitating the connection between the first luer connector 110 and the second luer connector 115. These luer connectors 110 and 115 are positioned at opposite ends of the tubular main body 105 and are configured to interface with the venous and arterial lines 10 and 20 via respectively mating patient luer connectors 11 and 21, enabling fluid communication for the priming portion of hemodialysis process. In this example, the connector 100 may be oriented in either direction along the flow path. That is, the first luer connector 110 may form a luer connection with the arterial patient connector 11 with the second luer connector 115 forming a luer connection with the venous patient connector 21, or, alternatively, the first luer connector 110 may form a luer connection with the venous patient connector 21 and the second luer connector 115 forming a luer connection with the arterial patient connector 11. It should be understood, however, that in some examples the connections may differ such that the connector 100 requires one orientation or the other.

As with the recirculation connector 30, recirculation connector 100 allows unobstructed fluid flow between the luer connections to allow the flow of priming fluid between the arterial and venous blood lines 10 and 20. However, in contrast to the recirculation connector 30, the recirculation connector 100 includes a clamping arm 140, which has an open position as shown in FIG. 2, and a closed position-as shown in FIGS. 4 to 6.

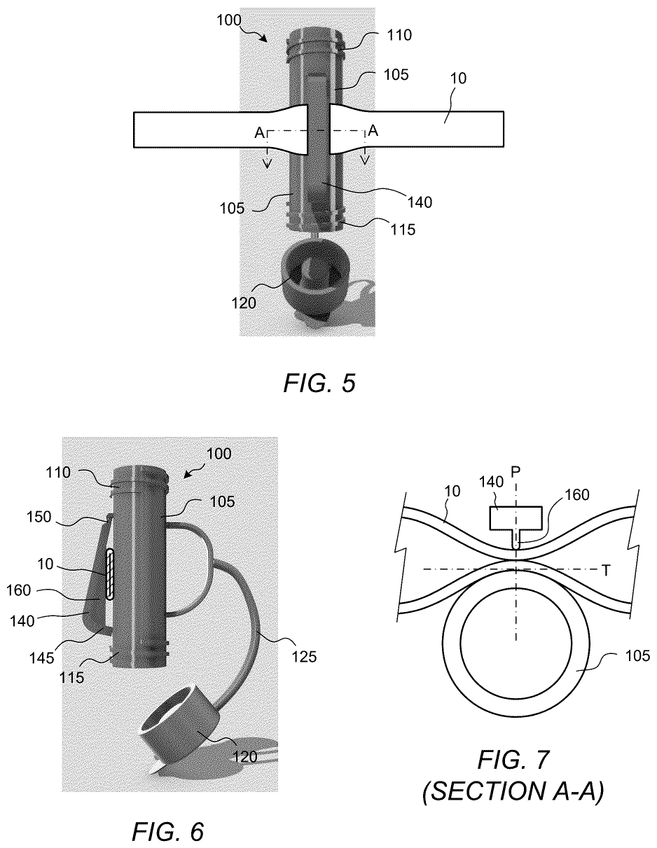

The clamping arm 140 has an open position, as shown in FIG. 2, and a closed position, as shown in FIGS. 4 to 6. When the clamping arm is in the open position, the connector 100 is configured to receive a tube (e.g., any portion of the line set 1 such as the arterial blood line 10 or venous blood line 20) between the clamping arm 140 and the tubular main body 105. With the tube positioned between the clamping arm 140 and the tubular main body 105, a user may press the clamping arm 140 from the open position to the closed position, thereby pinching and hermetically sealing the tube. This closed configuration is illustrated in FIGS. 5 and 6, showing arterial blood line 10 pinched closed between the clamping arm 140 and the tubular main body 105.

The clamping arm 140 includes a base 145 attached to the tubular main body 105, which functions as a pivot point about which the clamping arm 140 is rotated between the open position and the closed position. To maintain the clamping arm 140 in the closed position, the clamping arm includes a clamp latch 150 that engages with a corresponding catch 155 attached to the tubular main body to releasably lock the end of the clamping arm 140 opposite the base 145 in the closed position. The user may disengage the clamp latch 150 in this example by pushing the clamping arm laterally until the latch 150 disengages the catch 155 on the tubular main body 105. At this point, the connector 100 may be reused either at the same location or a different location, either during the same treatment or a subsequent treatment.

To increase focused clamping force across the clamped portion of the tube, the clamping arm 140 includes a clamping rib 160, which is narrower (in the context of the viewpoint of FIGS. 3 and 5 than the outwardly exposed portion of the clamping arm 140, which is the portion pressed by the user's finger or fingers to engage the clamping force. This rube 160 allows a high level of pressure to be focused along a line that extends transversely across the tube and extending across the entire flow path, resulting in the complete occlusion of the flow path as shown in FIG. 6.

The clamping action is shown in further detail in the cross-sectional view of FIG. 7. As shown, the clamping force is focused between the clamping rib 160 and the outer cylindrical surface of the tubular main body 105. This results in a pinching force that extends linearly along the length of the tubular main body 105 and the clamping rib 160 and exerted in a plane P that intersects the axis of the tubular main body 105 and is perpendicular to a plane T that is tangent to the outer surface of the tubular main body 105. Because the clamping rib 160 extends parallel to the axis of the tubular main body 105, the surfaces of contact between the connector 100 and the line 10 are stable and perpendicular to the tubular main body 105 along the length of contact.

The dual functionality of the connector 100 allows the device to perform both recirculation and occlusion tasks, streamlining the hemodialysis procedure and potentially eliminating the need for separate hemostats.

Additionally, the example of FIGS. 2 to 7 includes a luer cap 120 and cap tether 125, which provide protective coverage for the luer connection 115 when not in use, ensuring sterility and preventing contamination. The tether 125 prevents loss of the luer cap 120 when removed and ready availability when needed. Although this example includes a tethered luer cap, it should be understood that other examples may include an untethered luer cap or may not include a tether cap at all. Further, some examples may include a second luer cap, tethered or untethered, for the other luer connection 110. The example of FIGS. 2 to 5 may be suitable, for exampler, for inclusion in sealed/sterilized blood line sets where the first luer connection 110 is preattached to a respective end of the arterial blood line 10 or venous blood line 20. As such, the single cap 120 would seal the only remaining open end (i.e., luer connection 115) of the connector 100.

In some examples, the tubular main body 105 may be constructed of a single-use material, such as medical-grade plastic, which allows for easy disposal after a hemodialysis session, thereby maintaining hygiene standards and preventing cross-contamination. Alternative configurations might involve different materials or designs of the luer connections.

The ability of the clamping arm 140 to be repeatedly engaged and disengaged adds to its versatility and usability across multiple treatment sessions. In some alternative embodiments, the clamping arm 140 may be constructed from different materials or employ alternative locking mechanisms.

As indicated above, the connector 100 provides dual functionalities for hemodialysis treatment: a recirculation connection and tube occlusion. The recirculation connector 100 is utilized during the priming stage of hemodialysis treatment, where its primary function is to bridge the venous and arterial lines, allowing for the circulation of saline solution throughout the blood line tubing set. This circulation expels air from the fluid path before the initiation of treatment. The incorporation of a clamping feature into the recirculation connector 100 may eliminate the need for external hemostats to occluding portions of the tubing set 1. The positioning of the clamping arm 140 on the outer surface of the tubular main body 105 ensures that it is accessible and easy to engage. In some embodiments, the clamping arm 140 is designed to be repeatedly engaged and disengaged. Alternative examples might include variations in the occlusion mechanism, such as utilizing a different clamping force or mechanism.

As described above, the tubular main body 105 is the primary structural component of the recirculation connector 100. It integrates with the first luer connector 110 and the second luer connector 115, positioned at opposite ends of the tubular main body 105. These connections facilitate fluid communication between venous and arterial lines 10 and 20, ensuring efficient fluid transfer during hemodialysis priming. The tubular main body 105 in the illustrated example is an elongated cylindrical structure supporting other components and providing a conduit for fluid flow. It should be understood that other examples may provide differing geometries for the tubular main body, including non-cylindrical cross-sectional shapes. The first and second luer connectors 110 and 115 maintain the integrity of the fluid path, connecting securely to standard venous and arterial lines, such as lines 10 and 20 of FIG. 1.

In this regard, the luer connectors 110 and 115, integrally affixed at respective and opposed ends of the tubular main body 105, are configured to connect securely with corresponding luer connectors of the venous and arterial lines 10 and 20 in a male/female mating configuration, facilitating the role of fluid exchange. This connection is achieved through standardized fittings that comply with medical standards, such as ISO 80369-7, which provide a leak-free interface. Examples of luer connections include twist-lock or push-fit connectors designed for rapid engagement. In some examples, such as the recirculation connector 100 of FIGS. 2 to 7, the tubular main body 105 is integrally formed with the luer connectors 110 and 115, which in some examples may enhance structural integrity and reduce potential points of failure. It should be understood, however, that other examples could include modular designs where the luer connectors 110 and 115 are detachably connected to the tubular main body 105.

The clamping arm 140 of the illustrated example of a clamping portion. The term “clamping portion,” as used herein, refers to a segment of the device that applies pressure to a tube to restrict or control fluid flow. Some non-limiting examples of clamping portions include mechanisms such as snap-fit, lever-operated, or screw-based clamps, each offering a different method of engagement and release. The primary function of the clamping portion is to pinch a portion of the blood tube effectively, such as described above in connection with clamping arm 140, ensuring fluid occlusion. The clamping portion (in the illustrated example, clamping arm 140) is configured to engage securely and occlude the blood tube, preventing the introduction of air or leakage of blood during the procedure. This mechanism allows for control over the flow of blood or other fluids as part of the hemodialysis process.

As used herein, an element or operation recited in the singular and preceded with the word “a” or “an” should be understood as not excluding plural elements or operations, unless such exclusion is explicitly recited. References to “one” embodiment or implementation of the present disclosure are not intended to be interpreted as excluding the existence of additional embodiments that also incorporate the recited features. Furthermore, a description or recitation in the general form of “at least one of [a], [b] or [c],” or equivalent thereof, should be generally construed to include [a] alone, [b] alone, [c] alone, or any combination of [a], [b] and [c].

Implementations of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A method, comprising:
fluidly connecting a first end of an arterial blood line to a first end of a venous blood line via a recirculation connector, the arterial and venous blood lines part of an extracorporeal blood circuit of a dialysis machine;
flowing a priming fluid through the extracorporeal blood circuit such that the priming fluid either (a) flows from the arterial line to the venous line via the recirculation connector or (b) flows from the venous line to the arterial line via the recirculation connector;
after flowing the priming fluid through the extracorporeal blood circuit, removing the recirculation connector;
occluding a flow path in the extracorporeal blood circuit by using the recirculation connector to apply a clamping force to an outer surface of a portion of the arterial line or the venous line.

2. The method of claim 1, wherein the recirculation connector includes a clamping arm movable between an open position and a closed position, and the occluding includes moving the clamping arm from the open position to the closed position.

3. The method of claim 2, further comprising latching the clamping arm into the closed position while applying the clamping force.

4. The method of claim 3, further comprising:
unlatching the clamping arm to release the clamping force;
removing the recirculation connector from the portion of the arterial line or venous line; and
using the recirculation connector to apply a clamping force to another portion of the venous line or the arterial line.

5. The method of claim 2, wherein the recirculation connector includes a tubular body, and the clamping arm extends parallel to a longitudinal axis of the tubular body.

6. A medical fluid connector, comprising:
a tubular body having a first end, a second end, and an interior flow path extending along a longitudinal axis of the tubular body between the first end and the second end;
a first luer connector at the first end and configured to connect to an arterial line of an extracorporeal blood circuit;
a second luer connector at the second end and configured to connect to a venous line of an extracorporeal blood circuit; and
a clamping portion extending from an exterior surface of the tubular body and including a clamping rib, the clamping portion movable relative to the tubular body between an open position and a closed position, wherein
when the clamping portion is in the open position, the medical fluid connector is configured to receive a medical tube between the clamping rib and the exterior surface of the tubular body such that the medical tube is oriented transversely to the longitudinal axis of the tubular body, and
when the clamping portion is in the closed position, the clamping rib extends parallel to the longitudinal axis of the tubular body and the medical fluid connector is configured to occlude the medical tube by applying a clamping force to the medical tube between the clamping rib and the exterior surface of the tubular body.

7. The medical fluid connector of claim 6, further comprising a latch configured to maintain the clamping portion in the closed position.

8. The medical fluid connector of claim 7, wherein the latch comprises a protrusion on the first arm configured to engage with a corresponding recess on the second arm.

9. The medical fluid connector of claim 6, wherein the first luer connector is a female luer connector and the second luer connector is a female luer connector.

10. The medical fluid connector of claim 6, wherein the tubular body, the first luer connector, the second luer connector, and the clamping portion are integrally formed as a single monolithic molded component.

11. The medical fluid connector of claim 6, wherein the clamping portion is configured to be repeatedly opened and closed.

12. The medical fluid connector of claim 6, further comprising:
an end cap configured to cover the first luer connector or the second luer connector; and
a tether having a first end connected to the end cap and a second end connected to the tubular body.

13. An extracorporeal line set for dialysis, comprising:
a venous blood line;
an arterial blood line; and
a recirculation connector including
a tubular body having a first end, a second end, and an interior flow path extending along a longitudinal axis of the tubular body between the first end and the second end,
a first luer connector at the first end and configured to connect to the arterial line,
a second luer connector at the second end and configured to connect to the venous line, and
a clamping portion extending from an exterior surface of the tubular body and including a clamping rib, the clamping portion movable relative to the tubular body between an open position and a closed position,
wherein, when the clamping portion is in the open position, the medical fluid connector is configured to receive a portion of the venous line or a portion of the arterial line between the clamping rib and the exterior surface of the tubular body such that the portion of the venous line or the portion of the arterial line is oriented transversely to the longitudinal axis of the tubular body, and
wherein, when the clamping portion is in the closed position, the clamping rib extends parallel to the longitudinal axis of the tubular body and the medical fluid connector is configured to occlude the portion of the venous line or the portion of the arterial line by applying a clamping force to the portion of the venous line or the portion of the arterial line between the clamping rib and the exterior surface of the tubular body.
